(19) United States
(12) Patent Application Publication
(10) Pub. No.: US 2016/0214146 A1
(43) Pub. Date: Jul. 28, 2016

(54) Rotating Wash Group

(71) Applicant: BELIMED AG, Zug (CH)

(72) Inventor: Samo PETRIC, Stari trg pri Lozu (SI)

(73) Assignee: BELIMED AG, Zug (CH)

(21) Appl. No.: 14/917,758

(22) PCT Filed: Sep. 1, 2014
(86) PCT No.: PCT/IB2014/064162

§ 371 Date: Mar. 9, 2016

(30) Foreign Application Priority Data
Sep. 18, 2013 (SI) P-201300278

(51) Int. Cl. B08B 3/02; A61B 90/70

(52) U.S. Cl. CPC B08B 3/02; A61B 90/70

(57) ABSTRACT

The present invention relates to a washing machine in a sterilisation section of a hospital for washing and/or sterilisation of a surgical instruments, for instance, and similar, and in particular to an improved rotating wash group of said washing machine. It is provided for according to the present invention that said wash group comprises a tubular body (2) with an inlet projection (3) for a washing agent, a tubular washing rotor (4) having a set (11, 12) of through-holes (13) arranged in a rotatable manner inside the tubular body (2), and holding flanges (5, 6) to secure the rotor (4) in said body (2).

The present invention relates to a washing machine in a sterilisation section of a hospital for washing and/or sterilisation of a surgical instruments, for instance, and similar, and in particular to an improved rotating wash group of said washing machine.

The aforementioned washing machine is generally known, where a rotating wash group comprises a rotating nozzle arranged on a supporter inside the washing machine. During operation of said washing machine said nozzle rotates about its axis and sprays jets of water and/or washing agent over the instruments to be washed in the washing machine. The problem that occurs with said process is relatively deficient washing of the instruments in particular long and thin multi-joint instruments such as small pliers, for the jets of water and/or washing agent from the rotating nozzle do not reach all parts of the instruments to be washed and/or sterilised.

It is the object of the present invention to create a wash group particularly with a washing machine in a sterilisation section of a hospital for washing and/or sterilisation of a surgical instruments, for instance, and similar, which remedies drawbacks of know solutions.

The object as set above is solved with the characteristics disclosed in a characterizing clause of the claim 1. Detail of the invention is revealed in the corresponding subclaims.

The invention is further described in detail by way of non-limiting embodiment, and with a reference to the accompanying drawings, where

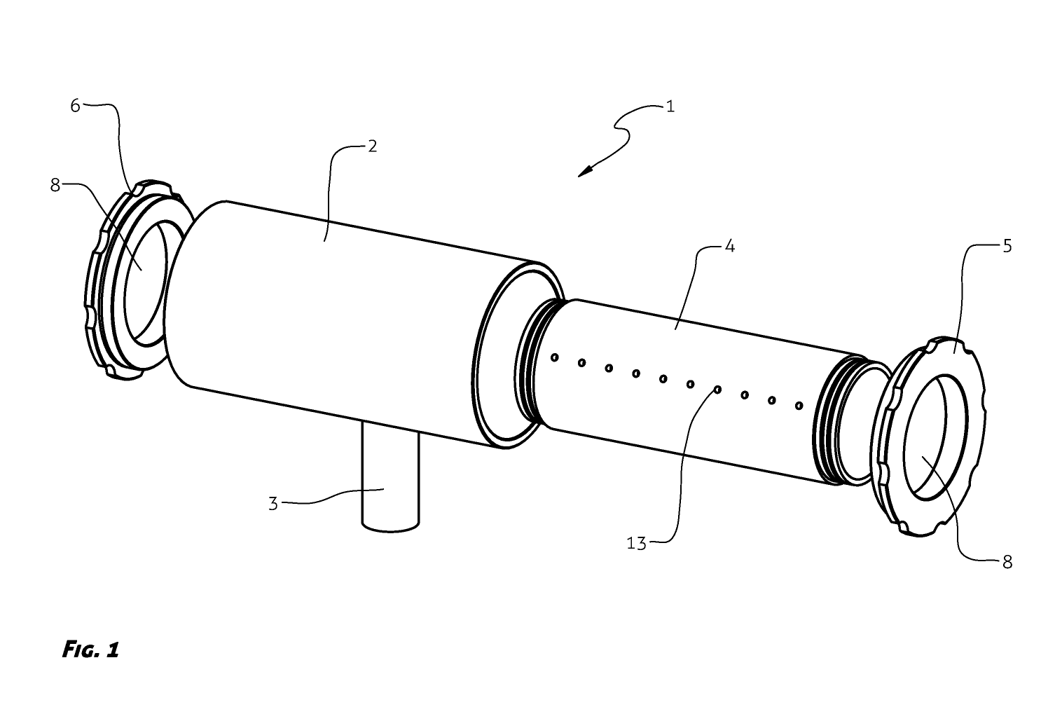

FIG. 1 shows a three-dimensional exploded view of a rotating wash group according to the invention,

FIG. 2 shows a cross-sectional view of the assembled rotating wash group of FIG. 1 through the longitudinal vertical plane,

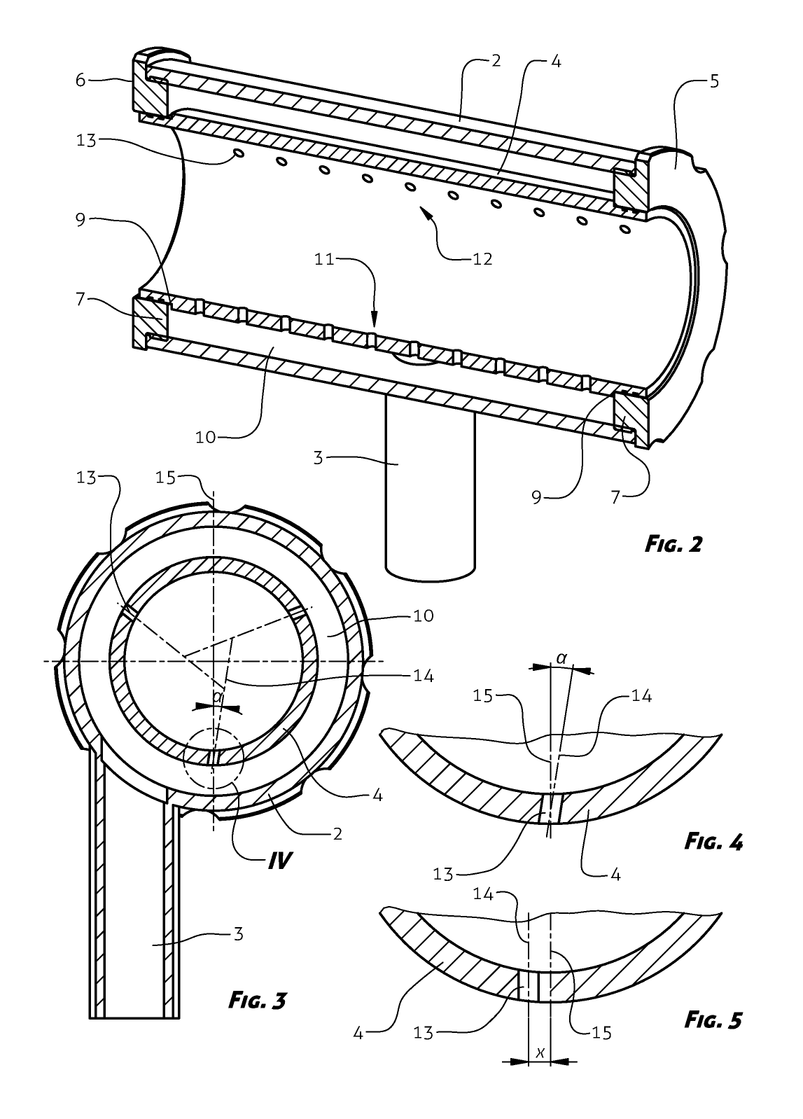

FIG. 3 shows a cross-sectional view of the assembled rotating wash group of FIG. 1 through the transversal vertical plane,

FIG. 4 shows detail IV of FIG. 3,

FIG. 5 shows another embodiment of detail of FIG. 4.

FIG. 1 shows a three-dimensional exploded view of a rotating wash group 1 of a washing machine in particular a washing machine in a sterilisation section of a hospital for washing and/or sterilisation of a surgical instruments, for instance, and similar, comprising a tubular body 2 with an inlet projection 3 for a washing agent, a tubular washing rotor 4 arranged in a rotatable manner in the body 2, and holding flanges 5, 6 to secure the rotor 4 in said body 2. Said body 2 and said rotor 4 are arranged approximately coaxial. Said inlet projection 3 is arranged eccentrically to the body 2, viewed in the transversal direction thereof. Each flange 5, 6 is formed with a centric radially extending cylindrical projection 7 provided with a centric through-hole 8. The external circumference of said projection 7 is connected by means of a temporary joint, such as a threaded joint, with each end of the body 2. Said tubular rotor 4 arranged in the body 2 is formed in the area of its each end with a radially extending step 9 with which cooperates each flange 5, 6 pressing against said step 9, thus, fixing the rotor 4 in the body 2. It is obviously, of course, that the connection between each step 9 of the rotor 4 and each flange 5, 6 is formed with a sliding fit in order for the rotor 4 to rotate in said flanges 5, 6. Thus, a substantial amount of space 10 is provided in the described manner between said body 2 and said rotor 4, said space 10 being filed with a washing agent during operation of the wash group 1 according to the invention.

Furthermore, said rotor 4 is formed over its circumference with at least two sets 11, 12 of through-holes 13 to supply the washing agent from said space 10 into the tubular rotor 4. Each said set 11, 12 of through-holes 13 lies approximately in the longitudinal direction of the rotor 4 with regard to the body axis of the rotor 4, however, other extends are possible such as in the sense of a helical curve, a spiral and the like. Each hole 13 is formed in the cross-sectional view of the assembly 1 (FIG. 3, FIG. 4) in a manner that a body axis 14 of the hole 13 is inclined for the angle α with regard to the transversal body axis 15 of the rotor 4, said angle α ranges between 15° in 45°. Further possibility provides a form of each hole 13 where its body axis 14 extends in parallel to the body axis 15 of the rotor 4 yet the body axis 14 of the hole 13 is formed so as to be spaced for the value x from the body axis 15 of the rotor 4 (FIG. 5). It is, however, obvious that also an arbitrary combination of the above described arrangement of the holes 13 is possible without departing from the spirit and scope of the invention.

During operation of a washing machine in particular a washing machine in a sterilisation section of a hospital for washing and/or sterilisation of a surgical instruments, for instance, and similar, the surgical instrument to be washed is inserted into the rotating wash group 1 according to the invention, in particular into the central section of the tubular rotor 4. Afterwards, the pressurized washing agent is dispensed via said inlet projection 3 into the space 10 between the body 2 and the rotor 4, said washing agent passes through the set 11, 12 of the through-holes 13 from the space 10 to the interior of the tubular rotor 4. Due to said inclination a of the holes 13 and, respectively, due to said spacing x of the holes 13, and due to said eccentric arrangement of the inlet projection 3 the washing agent that streams in exerts a tangential fluid load to the walls of the holes 13 resulting in a rotational movement of the rotor 4. The washing agent that streams via holes 13 in the interior of the rotor 4 sprays through said holes 13 and the resulting jets intersect in the central section of the rotor 4 and hit the surgical instrument to be washed over the entire length of the rotor 4 and, respectively, the length of the set 11, 12 of the holes 13. Thus, an excellent cleaning effect is achieved since the jets of the washing agent reach all the places on the surgical instrument to be washed.

1. An improved rotating wash group of a washing machine for washing and/or sterilisation of surgical instruments, comprising a tubular body with an inlet projection for a washing agent, a tubular washing rotor having a set of through-holes arranged in a rotatable manner inside the tubular body, and holding flanges to secure the rotor in said body.

2. The rotating wash group according to claim 1, wherein said rotor is formed over its circumference with at least two sets of through-holes to supply the washing agent from a space between the body and the rotor into the tubular rotor, each said set of the holes extends approximately in a longitudinal direction of the rotor with regard to the body axis of the rotor.

3. The rotating wash group according to claim 1, wherein each said set of holes extends in the a helical curve, a spiral or the like with regard to the body axis of the rotor.

4. The rotating wash group according to claim 1, wherein each hole is formed in a manner that its body axis, viewed in a cross-section of the group, is formed in a manner that its body axis is inclined for an angle a with regard to a transversal body axis of the rotor.

5. The rotating wash group according to claim 4, wherein the value of the angle is between approximately 15° and approximately 45°.

6. The rotating wash group according to claim 1, wherein each hole is formed in a manner that its body axis extends, in parallel with the body axis of the rotor, said body axis of the hole being formed so as to be spaced for the value x from the body axis of the rotor.

7. The rotating wash group according to claim 1, wherein said body and said rotor are arranged approximately coaxial.

8. The rotating wash group according to claim 1, wherein said inlet projection is arranged eccentrically with regard to the body when viewed transversally.
